(19) United States
(12) Patent Application Publication
(10) Pub. No.: US 2012/0157563 A1
(43) Pub. Date: Jun. 21, 2012

(54) WATER SOLUBLE SOLID PHASE PEPTIDE SYNTHESIS

(75) Inventor: Jonathan M. Collins, Charlotte, NC (US)

(73) Assignee: CEM CORPORATION, Matthews, NC (US)

(21) Appl. No.: 13/363,650

(22) Filed: Feb. 1, 2012

Related U.S. Application Data
(63) Continuation-in-part of application No. 13/209,960, filed on Aug. 15, 2011.
(60) Provisional application No. 61/373,989, filed on Aug. 16, 2010.
(60) Provisional application No. 61/382,550, filed on Sep. 14, 2010.
(60) Provisional application No. 61/441,390, filed on Feb. 10, 2011.
(60) Provisional application No. 61/469,881, filed on Mar. 31, 2011.

(51) Int. Cl. C07K 1/06; C08J 3/28; C08G 65/334

(52) U.S. Cl. 522/166; 530/336; 525/54.11

(57) ABSTRACT

A solid phase peptide synthesis method is disclosed. The method includes the steps of deprotecting an amino group in its protected form that is protected with a protecting group that includes an α,β-unsaturated sulfone; washing the deprotected acid in a solvent selected from the group consisting of water, alcohol, and mixtures of water and alcohol; coupling the deprotected acid to a resin-based peptide or a resin-based amino acid; and washing the coupled composition in a solvent selected from the group consisting of water, alcohol, and mixtures of water and alcohol.

### RELATED APPLICATIONS

This application is a continuation-in-part of Ser. No. 13/209,960 filed Aug. 15, 2011 for “Water Soluble Solid Phase Peptide Synthesis.” Ser. No. 13/209,960 claims priority from U.S. provisional application Ser. Nos. 61/373,989 filed Aug. 16, 2010; 61/382,550 filed Sep. 14, 2010; 61/441,390 filed Feb. 10, 2011 and 61/469,881 filed Mar. 31, 2011.

### BACKGROUND

The present invention relates to solid phase peptide synthesis (SPPS) and to a method of carrying out SPPS reactions in aqueous solutions.

Peptides are linked chains of amino acids which in turn are the basic building blocks for most living organisms. Peptides are also the precursors of proteins; i.e., long complex chains of amino acids. Peptides and proteins are fundamental to human and animal life, and they drive, affect, or control a wide variety of natural processes. As a result, the study of peptides and proteins and the capability to synthesize peptides and proteins are of significant interest in the biological sciences and medicine.

Solid phase peptide synthesis is a technique in which an initial amino acid is linked to a solid particle and then additional amino acids are added to the first acid to form the peptide chain. Because the chain is attached to a particle, it can be washed and otherwise treated with additional solvents or rinses while being maintained in a discrete vessel and handled (at least to some extent) as a solid. SPPS thus allows solution phase chemistry to be carried out in a manner that has some of the convenience of handling solids.

Conventional SPPS is most typically carried out in polar organic solvents such as dimethyl formamide (DMF), n-methylpyrrolidone (NMP), dimethyl sulfoxide (DMSO) and dichloromethane (DCM). DCM is typically mixed with DMF or NMP because the N-alpha protecting groups Fmoc (e.g., fluorenylmethyloxycarbonyl chloride) and Boc (e.g., tert-butoxycarbonyl) frequently used in SPPS are typically hydrophobic and insoluble in water. Although Fmoc and Boc (e.g., tert-butoxycarbonyl) synthesis methods have had a major impact on SPPS they both suffer from their need for organic solvents that are costly and toxic.

These toxic solvents require the use of special laboratory techniques, such as carrying out the reactions entirely under a fume hood or equivalent device. Fume Hood space is limited and thus valuable in the laboratory context. As a result, SPPS using these solvents is expensive from a landscape standpoint.

These organic solvents tend to be aggressive and require upgraded equipment. Their disposal represents an environmental hazard and at a minimum is regulated.

In conventional SPPS, the Fmoc group is removed by a secondary amine (piperidine, piperazine, morpholine) in a 6-elimination reaction during SPPS. An undesirable feature of this mechanism is that it generates a reactive dibenzofulvene (DBF) that is scavenged by (e.g.) excess piperidine. The DBF can, however, also react with the free amine group effectively capping the end of the peptide chain. Some deprotection employ a short initial deprotection step to flush most of the DBF out of the reaction vessel and then use a second longer deprotection with fresh piperidine solution to reduce this potential side reaction. This approach may be unnecessary, however, because a typical 20% deprotection solution has a large excess of piperidine versus potential DBF. For example, a synthesis at 0.1 mmol scale using a 7 mL solution of a 20% piperidine in DMF would have a ratio of piperidine to total potential DBF of approximately 710:1.

Based upon these and other factors, an aqueous based—i.e., water-soluble—scheme for peptide synthesis, and particularly SPPS, represents a worthwhile ongoing technological goal.

As one attempt, some authors have hinted that finely powdered or pulverized reagents can increase the water solubility of the relevant SPPS compositions, but such results are to date difficult to confirm or reproduce.

As another attempt, Galanis (Organic Letters, Vol. 11, No. 20, pp. 4488-4491 (2009)) has used a conventional Boc protecting group in the presence of specific resins, linkers, activating agents and a zwitterion detergent to produce a single demonstrative Leu-Enkephalin peptide.

As a more promising option, water soluble protecting groups have been attempted. Hojo (Hojo et al; Chem. Pharm. Bull. 52, 422-427 2004; Hojo, K.; Maeda, M.; Kawasaki, K. Tetrahedron Lett. 45, 9293 2004) has developed several protecting groups for this purpose that include 2-(Phenyl(methyl)sulfoniol)ethyloxy carbonyl tetrafluoroborate (Pms), Ethanesulfonylethoxycarbonyl (Esc), and 2-(4-Sulfophenylsulfonyl)ethoxy carbonyl (Sps).

These reports are, of course, exemplary rather than comprehensive.

Although amino acids carrying these protecting groups are water-soluble, the groups raise other difficulties that make their routine use more difficult. The Pms group is an onium salt and thus significantly less stable than conventional protecting groups. Esc is more stable than Pms and offers moderate aqueous solubility. The starting material, however, for the Esc group is relatively expensive. Additionally, the Esc-Cl group is unstable and the group must be converted to ethanesulfonylethyl-4-nitrophenyl carbonate (ESC—ONp) for use with amino acids.

Sps has a solubility comparable to that of Esc, but synthesizing Esc appears to be more complicated and expensive. Additionally, a different synthesis scheme must be used for cysteine (Cys) and methionine (Met) in order to avoid oxidation of their sulfur groups.

As a secondary consideration, a larger number of aromatic rings in a protecting group molecule can enhance the UV absorption for conventional monitoring purposes. The additional rings, however, also minimize or eliminate water solubility.

In conventional monitoring methods, a reaction product is drawn after the deprotection step and measured under UV absorption. Fmoc will absorb characteristic UV frequencies (e.g., 300 nanometers) in amount proportional to its concentration and thus the amount of detected Fmoc will provide an indication of the extent to which deprotection has proceeded

Because of their molecular structure, Pms, Esc, and Sps have the advantage of some water solubility, but Pms and Esc cannot be tracked in conventional UV monitoring in the same manner as conventional Fmoc. Sps can be monitored by UV, but its difficult and costly synthesis tends to discourage its use. As a result, the increased water solubility of these compounds is less helpful in an overall sense.

Nevertheless, these compositions tend to produce low purity peptides at each step thus limiting overall peptide length. Also, many amino acids used in SPPS include side chain protecting groups (e.g., trityl “Trt” and t-butyl “tBu”) which are hydrophobic. Thus, the solubility in an aqueous environment continues to decrease as such acids are added to the chain.

As another disadvantage, the activated species tend to get hydrolyzed in water and will no longer react with the growing peptide chain.

Continued work with water soluble protecting groups also shows that peptide chains tend to aggregate more in the polar solvents (water, alcohol) than in the conventional organic solvents (DMF, NMP). Because such aggregation reduces reactivity at each step, its effects are multiplied over the multiple steps of a peptide synthesis.

Additionally, when water is present during deprotection and coupling, particular side reactions tend to occur. When water is present with a strong base—for example piperidine used for the deprotection reaction—the strong base tends to deprotonate the water and produce hydroxide ions. In turn, the presence of a hydroxide ion tends to generate undesired racemization. Additionally, water present during the coupling reaction can hydrolyze amino acids, which renders the hydrolyzed acids incapable of coupling with the growing peptide chain.

Therefore, a need continues to exist to increase the use of aqueous-based systems during peptide synthesis in general and solid phase peptide synthesis in particular.

### SUMMARY

The invention is an improvement in solid phase peptide synthesis that includes deprotecting an amino acid and then washing the deprotected acid in a solvent selected from the group consisting of water, alcohol, and mixtures of water and alcohol.

In exemplary aspects, the invention includes the steps of deprotecting an amino group in its protected form that is protected with a protecting group containing an alpha, beta (α,β) unsaturated sulfone and then washing the deprotected acid in a solvent selected from the group consisting of water, alcohol, and mixtures of water and alcohol.

In other aspects, the amino acid is protected with a protecting group that acts as a Michael Reaction acceptor in the presence of a Michael Reaction donor

In exemplary aspects, the protecting group is selected from the group consisting of Bsmoc, Nsmoc, Bspoc and Mspoc; and with Bsmoc being typical.

In another aspect, the invention is a solid phase peptide synthesis method that includes the improvement of deprotecting an amino acid that is soluble in aqueous environments in its protected form, and then washing the deprotected acid in a solvent selected from the group consisting of water, alcohol, and mixtures of water and alcohol

In another aspect, the invention is a solid phase peptide synthesis method that includes the improvement of deprotecting a Bsmoc-protected amino acid, and then washing the deprotected acid in a solvent selected from the group consisting of water, alcohol, and mixtures of water and alcohol.

In another aspect, the invention is a solid phase peptide synthesis method that includes the steps of coupling a protected acid to a resin-based peptide or a resin-based amino acid; and washing the coupled composition in a solvent selected from the group consisting of water, alcohol, and mixtures of water and alcohol.

In another aspect, the invention is a composition that includes a solid phase resin, an amino acid protected with a protecting group containing an α,β-unsaturated sulfone, a solvent selected from the group consisting of water, alcohol, and mixtures of water and alcohol, a base for deprotecting the protected amino acid, and the adduct formed by the reaction between the deprotecting base and the α,β-unsaturated sulfone protecting group.

In another aspect, the invention is a composition that includes a solid phase resin, a solvent selected from the group consisting of water, alcohol, and mixtures of water and alcohol, an unactivated α,β-unsaturated sulfone based protected amino acid, and activated portions of the α,β-unsaturated sulfone based protected amino acid.

In another aspect, the invention is a process for accelerating the solid phase synthesis of peptides by carrying out one or more of the steps of deprotecting an amino group in its protected form that is protected with a protecting group containing an α,β-unsaturated sulfone and linked to solid phase resin particles by admixing the protected linked acid with a deprotecting solution while irradiating the admixed acid and solution with microwaves; activating a second amino acid by adding the second acid and an activating solution; coupling the second amino acid to the first acid while irradiating the composition with microwaves; and successively deprotecting, activating, and coupling a plurality of amino acids into a peptide.

The foregoing and other objects and advantages of the invention and the manner in which the same are accomplished will become clearer based on the followed detailed description taken in conjunction with the accompanying drawings.

### BRIEF DESCRIPTION OF THE DRAWINGS

FIG. 1 illustrates a conventional Boc deprotection.

FIG. 2 illustrates a conventional Fmoc deprotection.

FIG. 3 illustrates the alternative pathways for Fmoc being scavenged by a base and as reacting with a non-protected amino acid to cap a peptide chain.

FIG. 4 illustrates an α,β-unsaturated sulfone protecting an amino acid.

FIG. 5 illustrates a Bsmoc deprotection according to the present invention.

FIG. 6 is the output plot of an HPLC chromatograph separation of the products of a peptide synthesis incorporating aspects of the present invention.

FIG. 7 is a mass spectrum of a relevant fraction from the HPLC chromatograph represented by FIG. 6.

FIG. 8 is the output plot of an HPLC chromatograph separation of the products of another peptide synthesis incorporating aspects of the present invention.

FIG. 9 is a mass spectrum of a relevant fraction from the HPLC chromatograph represented by FIG. 8.

FIG. 10 is the output plot of an HPLC chromatograph separation of the products of another peptide synthesis incorporating aspects of the present invention.

FIG. 11 is a mass spectrum of a relevant fraction from the HPLC chromatograph represented by FIG. 10.

FIG. 12 is the output plot of an HPLC chromatograph separation of the products of another peptide synthesis incorporating aspects of the present invention.

FIG. 13 is a mass spectrum of a relevant fraction from the HPLC chromatograph represented by FIG. 12.

### DETAILED DESCRIPTION

In a broad aspect, the invention is a solid phase peptide synthesis method in which the improvement comprises using one or more amino acids that are protected with a protecting group includes an α,β-unsaturated sulfone. Typically, such a protecting group acts as a Michael Reaction acceptor in the presence of a Michael Reaction donor. In the invention, the washing steps are carried out in a solvent selected from the group consisting of water, alcohol, and mixtures of water and alcohol.

It has now been determined that an organic solvent system is (i.e., continues to be) advantageous for the deprotection and coupling steps, but that significant advantages can be obtained by carrying out the washing steps in an aqueous environment, including solvent systems that include water, or polar alcohols, or mixtures of water and alcohols. For purposes of clarity (but not limitation) such solvent systems (water, alcohol, or water and alcohol) will be referred to herein as “aqueous” solvent systems.

In particular, over 90% of the organic solvents used in conventional SPPS are used during the washing steps. Therefore, carrying out the deprotection and coupling steps in more favorable organic solvents while replacing organic solvents during the washing steps, has the potential to replace 90% of the organic solvents without sacrificing productivity

The invention represents the recognition (in part) that the washing steps can be carried out in water, or a polar alcohol, or an alcohol and water solvent system, if the N alpha protecting group incorporates an alpha, beta unsaturated sulfone system.

Additionally, the invention represents the recognition that effective washing requires that the solid phase resin swell adequately in water (or the water-alcohol solvent system) to effectively ensure complete washing. In that regard, it appears that the polyethylene glycol (PEG) solid phase resins swell better in the water-alcohol or water solvent systems then do the polystyrene-based resins.

In exemplary aspects, the protecting group is selected from the group consisting of Bsmoc, Nsmoc, Bspoc and Mspoc; and with Bsmoc being typical.

As well understood by the skilled person, a Michael Addition reaction is the nucleophilic addition of a nucleophile to an alpha, beta unsaturated carbonyl compound. The nucleophile is the Michael Donor (e.g., piperidine) and the alpha, beta unsaturated carbonyl compound is the Michael Acceptor (e.g. an alkene).

In exemplary embodiments of the present invention, the amino acid protecting group has a Michael acceptor site that includes an alpha, beta-unsaturated sulfone.

The use of the alpha, beta unsaturated sulfone system for the protecting group offers at least two advantages in an aqueous-based washing step. First, the sulfone functional group is extremely soluble in water. This in turn greatly increases the solubility of amino acids that use this class of protecting groups.

As is well understood in the art, in order to be effective, particularly for large numbers of cycles, the washing steps must remove all of the unreacted amino acids and all of the excess protected amino acids from the reaction vessel. In comparison to the alpha, beta unsaturated sulfone protecting groups, Fmoc protected acids are highly insoluble in water and are not removed by an aqueous washing step.

As a second advantage, the alpha, beta unsaturated sulfone compositions have a higher base lability than do Fmoc protected amino acids. As a result, the alpha, beta unsaturated sulfone protecting groups can be removed by a more reactive Michael addition mechanism (as opposed to the beta-elimination mechanism used to remove Fmoc). This higher lability permits a correspondingly lower amount (concentration) of the base to be utilized for the deprotection step. Additionally, weaker bases such as piperazine (pKa=9.83) can be more effectively substituted for stronger bases such as piperidine (pKa=11.123). In either case (a lesser concentration of a strong base or an equivalent concentration of a weaker base) the moderated base (i) reduces or effectively eliminates the undesired aspartimide formation that tends to occur during either the next deprotection step or a subsequent washing step when small amounts of the base remain behind, and (ii) minimizes or eliminates undesired formation of hydroxide ion (OH⁻) in any of the aqueous based steps or environments.

It will also be understood that as used herein, a phrase such as “soluble in water in its protected form” means that the composition has the degree of solubility necessary for the desired reaction to proceed in an aqueous solvent system. As is the case with any composition, the term “soluble” does not imply unlimited solubility in any or all amounts.

As used herein, the abbreviation Bsmoc refers to 1,1-dioxobenzo[b]thiphene-2-ylmethyloxycarbonyl. Bsmoc is also referred to by the “common name” benzo[b]thiophenesulfone-2-methyloxycarbonyl. Bsmoc is typically represented by the following formula:

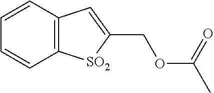

An early discussion of Bsmoc as a protecting group for amino acids during SPPS synthesis is set forth by Carpino et al in the Journal or Organic Chemistry, 1999, 64 (12) at pages 4324-4338.

Four of the standard Bsmoc amino acid derivatives are difficult to handle at room temperature [Bsmoc-Asp(OtBu)-OH, Bsmoc-Leu-OH, Bsmoc-Pro-OH, Bsmoc-Ser(tBu)-OH] because they are either oils or have a low melting point (Asp—m.p. ˜43° C.). The 16 other Bsmoc derivatives are solids at room temperature with melting temperatures greater than 90° C. Therefore, for the four Bsmoc derivatives that are more difficult to handle the use of a higher molecular weight derivative Nsmoc (e.g., 1,1-dioxonaptho[1,2-b]thiophene-2-methyloxycarbonyl; “α-Nsmoc”) is recommended.

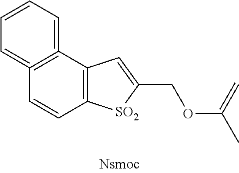

Nsmoc derivatives of all 20 standard amino acids have been successfully made and used in SPPS. The Nsmoc group shows similar advantages to the Bsmoc group, but appears somewhat more expensive to produce because of its additional six member carbon ring. The Nsmoc group is also predicted to result in a lower acylation rate than the Bsmoc group, but comparable to the Fmoc group because of their similar size. As a further possibility (and as known to the skilled person), two other Nsmoc isomers can be produced; i.e., with the second aromatic ring in a different position with respect to the SO₂ group.

Related α,β-unsaturated sulfone protecting groups that can function as the Michael acceptor include 2-tert-butylsulfonyl-2-propenoxycarbonyl (Bspoc) and 2-methylsulfonyl-3-phenyl-1-prop-2-enyloxycarbonyl (Mspoc); see, e.g., Carpino et al., The 2-methylsulfonyl-3-phenyl-1-prop-2-enyloxycarbonyl (Mspoc) Amino Protecting Group, J. Org. Chem. 1999, 64, 8399-8401.

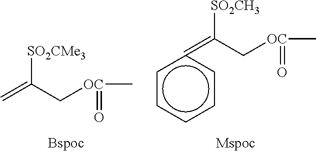

As a general point, the basic aspects of SPPS are generally well-understood in the art and by the skilled person, with the seminal work being carried out by Merrifield (R. B. Merrifield (1963), “Solid Phase Peptide Synthesis. I. The Synthesis of a Tetrapeptide”, Journal of the American Chemical Society 85 (14): 2149). Thus, SPPS details will not necessarily be repeated in detail herein and the skilled person can carry out the SPPS steps described herein without undue experimentation.

It will be understood that one of the advantages of the invention is the capability to carry out the washing steps using water, alcohol, or a water-alcohol mixture.

It will also be understood that the choice of washing solvent as between and among water, alcohol, and water-alcohol mixtures (as well as the water:alcohol ratio of any given mixture) will depend to some greater or lesser extent upon the amino acids desired for the target peptide, or the base selected for deprotection, or a combination of these factors. The straightforward nature of the invention enables the skilled person to make the selection on a case-by-case basis and without undue experimentation.

In exemplary embodiments, the method can also include irradiating the acid and the solvent with microwaves during the deprotection step. A detailed description of an instrument suitable for microwave irradiation is the SPPS context is set forth in commonly-owned U.S. Pat. No. 7,393,920 (and in a number or related patents and published applications), the contents of which are incorporated entirely herein by reference.

In some embodiments, the protected amino acid is one of the essential amino acids that remains sufficiently water-soluble (in both its activated and protected forms) when protected with the relevant protecting group to wash with the water (or water-alcohol) solvent system of the invention; e.g. an amino acid protected with Bsmoc. In this embodiment water is used as a solvent and a base that is soluble in water is used in an amount and to the extent necessary to deprotect the acid.

In general, a favorable base will be one for which the base and its deprotection adduct can be successfully removed (washed) in the aqueous-based solvent system of the present invention.

In accordance with appropriate peptide synthesis, the method can comprise repeating the steps of deprotecting, washing, coupling, and washing for a second protected acid. Thereafter, the steps can be repeated to add a third protected amino acid, and thereafter a successive plurality of protected amino acids to produce a desired peptide.

In another aspect, the invention is a method of solid phase peptide synthesis in which the improvement includes the steps of deprotecting an amino group in its protected form that is protected with a protecting group containing a Michael acceptor site composed of an α,β-unsaturated sulfone, and then washing the deprotected acid in a solvent selected from the group consisting of water, alcohol, and mixtures of water and alcohol. In this embodiment, the advantages of the water or alcohol or mixture solvent system can be used for the washing step independently of whether or not the solvent system is used for the deprotection step.

In exemplary embodiments the acid is protected with a protecting group selected from the group consisting Bsmoc, Nsmoc, Bspoc and Mspoc, with a Bsmoc-protected amino acid being most typical.

As in the case of the deprotection step, the washing step can be carried out in the presence of microwave irradiation on an as-needed or as-desired basis. When the washing step is carried out in the mixture of water and alcohol the alcohol again can be selected from the group consisting of methanol, ethanol, 1-propanol, 2-propanol, n-butanol, isobutanol, sec-butanol, and tert-butanol.

In yet another aspect, the invention is a method of solid phase peptide synthesis comprising deprotecting an amino group in its protected form that is protected with a protecting group containing a Michael acceptor site composed of an α,β-unsaturated sulfone, coupling the deprotected acid to a resin-based peptide or a resin-based amino acid, and then washing the coupled composition in a solvent selected from the group consisting of water, alcohol, and mixtures of water and alcohol. As was true with respect to the other steps in the process, the use of the water, alcohol or mixture solvent system can be in some cases limited to the step of washing the coupled composition and does not required that the deprotection or the coupling steps themselves be carried out in the same solvent system.

Bsmoc, Nsmoc, Bspoc and Mspoc protected amino acids are again exemplary.

The step of washing the coupled composition can likewise be enhanced in some circumstances by the use of microwave irradiation. The alcohols used for the water-alcohol mixture solvent system can be those mentioned previously and the bases used to deprotect the protected amino acids can be those bases named previously.

In another aspect, the invention is a solid phase peptide synthesis method that includes the following steps: deprotecting an amino group in its protected form that is protected with a protecting group containing a Michael acceptor site composed of an α,β-unsaturated sulfone; washing the deprotected acid in a solvent selected from the group consisting of water, alcohol, and mixtures of water and alcohol; coupling the deprotected acid to a resin-based peptide or a resin-based amino acid; and washing the coupled composition in a solvent selected from the group consisting of water, alcohol, and mixtures of water and alcohol.

As in other embodiments, Bsmoc, Nsmoc, Bspoc and Mspoc protected amino acids are again exemplary.

In order to enhance the reaction, microwaves can be applied during the deprotection step or the coupling step, including the steps of coupling single acids together or the step of coupling a sequential acid to a resin-based peptide or a resin based amino acid.

As in the previous embodiments, appropriate alcohols can include methanol, ethanol, 1-propanol, 2-propanol, n-butanol, isobutanol, sec-butanol, and tert-butanol.

Any appropriate base can be used to deprotect the relevant amino acids, but in exemplary embodiments, including Bsmoc-protected acids, weaker bases (e.g., piperazine, morpholine) can help minimize hydroxide (OH) concentration in an aqueous environment (and thus minimize racemization). Also, a single alcohol wash step prior to coupling appears to likewise help minimize hydrolysis of the activated amino acid being added by minimizing water present during the coupling step (i.e., activated amino acids are known to be susceptible to hydrolysis in water).

The deprotecting, coupling and washing steps can be repeated to add a second amino acid that is likewise initially protected with Bsmoc to the first amino acid. The steps can be repeated for a third and thereafter successive plurality of Bsmoc-protected acids to form a peptide chain.

The method can further include the step of cleaving the peptide chain from the solid phase resin, and microwave radiation can be applied to enhance the cleaving step.

In another aspect, the invention is a composition. In this aspect, the composition comprises a mixture of a solid phase resin and a solution. The solution comprises an amino acid and an amino acid protecting group, both dissolved in the same solvent. The protecting group includes an α,β-unsaturated sulfone, which in exemplary embodiments acts as a Michael Reaction acceptor in the presence of a Michael Reaction donor. The solvent is selected from the group consisting of water, alcohol, and mixtures of water and alcohol.

In exemplary embodiments, the composition further comprises a base that is soluble in the solvent system. In particular embodiments, the base is soluble in water alone. Water soluble bases appropriate for the composition include mild alkyl hydroxide bases, sodium hydroxide, lithium hydroxide, sodium carbonate, piperidine, 4-(Amino methyl)piperidine and piperazine.

In exemplary embodiments, Bsmoc (or Nsmoc, Bspoc or Mspoc) and an amino acid are dissolved in the same solvent.

The alcohol in the composition can in exemplary embodiments be selected from the group consisting of methanol, ethanol, 1-propanol, 2-propanol, n-butanol, isobutanol, sec-butanol, and tert-butanol.

In exemplary embodiments, the solid phase resin includes polyethylene glycol, either as the resin itself or as ethylene-oxide spacer groups between a polystyrene resin and its functional groups.

Additionally, the invention represents the recognition that effective washing requires that the solid phase resin swell adequately in water (or the water-alcohol solvent system) to effectively ensure complete washing. In that regard, it appears that the polyethylene glycol (PEG) solid phase resins swell better in the water-alcohol or water solvent systems then do the polystyrene-based resins.

As is generally well understood in/by skilled persons in SPPS, the swelling of the resin is an important factor because the reaction kinetics are controlled by the diffusion. As a result, resin that swells more (comparatively) will demonstrate a higher diffusion rate of the reagents into the core of the resin matrix. In turn, this results in shorter reaction times and more complete chemical conversions.

For example, 1% cross-linked polystyrene resins will demonstrate a swelling factor (original size to solvated size) of at least 3 in some organic solvents and greater than 5 in other solvents. On a proportional basis, polystyrene resins do not swell in water (i.e., swelling factor equal 1.0) and have a swelling factor of less than two in methanol.

In comparison, polyethylene glycol resins tend to show greater swelling in all solvents (polar and nonpolar) then do polystyrene resins. According to publications of Sigma Aldrich (St. Louis Mo. USA), certain PEG resins will demonstrate swelling factors greater than 10 in water. In the context of the present invention, the H-Rink Amide-ChemMatrix® resin from PCAS BioMatrix Inc. of Québec Canada is particularly favorable.

Even PEG “spacer” resins offer improved performance as compared to conventional polystyrene resins. As recognized by those of skill in this art, a spacer resin includes a polystyrene matrix, but also includes several (typically between 5 and 10) ethylene oxide units between the polystyrene matrix and the reactive sites. The spacer groups help modify the hydrophobic properties of the polystyrene backbone and the spacer resins demonstrate a higher mobility which in turn increases the rate of reaction.

In some embodiments, the aqueous solvent system can be enhanced by the presence of a detergent to help render a protected acid soluble in the aqueous-based solvent system. The term “soluble” is used herein in its usual sense; i.e., the desired or necessary amount of protected acid will completely dissolve in the solvent system. Persons of ordinary skill in the chemical arts will recognize, of course, that solubility is a relative term that can also be quantified based on the amount of a particular material that will dissolve in a particular solvent. Thus, for purposes of the invention, the respective compositions are considered soluble if they will dissolve in water in the amounts typically required to successfully carry out solid phase peptide synthesis.

Because the progress of deprotection reactions can be monitored on a periodic sample basis using an ultraviolet measurement of the amount of protecting group in solution, a detergent that avoids interfering with the UV absorption of the protecting group at the wavelengths characteristic of the protecting group can be helpful in the monitoring context.

Detergents are water soluble molecules classified according to their hydrophilic or hydrophobic character (or the degree of each) and their ionic groups. These characteristics establish the behavior of the detergent with respect to the protecting groups, the peptide chain, and individual amino acids.

In many cases a detergent has a hydrophobic tail that associates to form micelles, or that aggregates, or interacts with other molecules (lipids, proteins). In solution, detergents help keep molecules in solution by dissociating aggregates, and unfolding larger molecules

Typical detergents that are helpful include nonionic detergents, cationic detergents, anionic detergents, and zwitterionic detergents. Particular detergents that are useful include octyl phenyl ethylene oxide; sodium lauryl sulfate; and sodium dodecyl sulfate.

As an another advantage, it has been discovered that the use of a single alcohol wash before, or after, or both before and after the deprotection step helps ensure that the base and water are never present in significant proportions at any time. This is particularly useful when utilizing a more aggressive base (e.g., piperidine). By comparison, the use of the weaker base (piperazine) makes the single alcohol washing step less necessary.

It has also been discovered that the single alcohol wash prior to the coupling reaction enhances the overall synthesis quality by minimizing or eliminating the presence of water during coupling, which in turn helps protect the activated amino acid from hydrolysis.

In a manner consistent with conventional SPPS, the method can include activating the deprotected acid with an activator. Any activator that carries out the appropriate advantages (i.e. making the oxygen a better leaving group) and that otherwise is consistent with the overall SPPS reaction is appropriate. Representative activating agents include carbodiimides and triazoles. Other conventional activating agents can include O-Benzotriazolyl-N,N,N′,N′-tetramethyluronium hexafluorophospate (HBTU), 2-(1H-Benzotriazole-1-yl)-1,1,3,3-Tetramethyluronium Tetrafluoro Borate (TBTU), Boc-histidine(tosyl); BOP and BOP-Cl.

In yet another aspect, the invention is a process for accelerating the solid phase synthesis of peptides by carrying out one or more of the steps of deprotecting the alpha amino group of an amino acid in its protected form that is protected with a protecting group containing an α,β-unsaturated sulfone and linked to solid phase resin particles by admixing the protected linked acid with a deprotecting solution while irradiating the admixed acid and solution with microwaves. The method includes activating a second amino acid and then coupling the second amino acid to the first amino acid while irradiating the composition with microwaves. Thereafter the method includes successively deprotecting, activating, and coupling a plurality of amino acids into a peptide.

In exemplary embodiments, the amino acid is protected with Bsmoc, Nsmoc, Bspoc or Mspoc.

An instrument suitable for use in the method is described in detail in commonly assigned U.S. Pat. No. 7,393,920. The same description is set forth in other commonly assigned U.S. patents resulting from divisional and continuing applications and has also been published in Europe, for example at EP 1 491 552 and EP 1 923 396. These descriptions provide the skilled person with the information helpful to practicing the method.

The method can comprise cyclically repeating the steps of deprotecting, activating, and coupling for three or more amino acids in succession to thereby synthesize a desired peptide.

When the peptide (intended or desired) is complete, any of the methods described herein typically comprises cleaving the linked peptide from the solid phase resin by admixing the linked peptide with the cleaving composition. In some embodiments cleavage is carried out while irradiating the composition with microwaves.

As recognized by the skilled person, the cleaving compositions and protocol are to some extent dictated by the amino acids in the peptide chain and in some cases by the side protecting groups that those amino acids may carry. In most cases, an acid is used to carry out the cleaving step. In general, the acid should carry out the necessary cleavage without adversely affecting or interfering with the desired peptide and any desired groups (e.g., side chain protecting groups) that are attached to the amino acids in the peptide.

Trifluoroacetic acid and hydrofluoric acid (HF) are common cleaving agents, but are often mixed with small proportions of complementary compositions such as water, phenol and ethanedithiol (EDT). Trifluoromethane sulfonic acid (TFMSA) or trimethylsilyltrifluoromethanesulfonate (TMSOTf) are used as cleaving agents in some cases. These are, of course, exemplary rather than limiting of the cleaving composition possibilities. The cleaved peptide (in solution) can be separated from the cleaved resin by filtration and the peptide can then be recovered from the filtrate by a conventional step such as evaporation or solvent-driven precipitation.

Cleavage is typically carried out in the presence of scavenger compositions (e.g., water, phenol, EDT) which protect the peptide from undesired side reactions during and after the cleaving step. As recognized by the skilled person, the scavengers are generally selected based upon the protecting groups that are present. Thus, the selection is to some extent customized by the skilled person, who can select the appropriate scavengers without undue experimentation.

Synthesis of Bsmoc

Bsmoc can be synthesized using several reaction pathways. In an exemplary reaction, Bsmoc is formed from commercially available 1-benzothiophene through hydroxymethylation followed by peracid oxidation. The starting material 1-benzothiophene is readily available at modest pricing.

Elimination vs. Michael Addition Mechanism

In an exemplary embodiment of the invention, the protecting group (e.g., Bsmoc) is removed by a Michael Addition mechanism with a secondary amine. As noted previously, a Michael Addition reaction is the nucleophilic addition of a nucleophile to an electrophilic α,βunsaturated compound. The nucleophile is the Michael Donor (e.g., piperazine) and the α,β unsaturated compound is the Michael Acceptor (e.g. an alkene).

The protecting groups developed by Carpino (Bsmoc, Mspoc, Bspoc, Nsmoc) contain a Michael Acceptor group. The Michael Acceptor group for these compounds is an activated alkene group. A Michael Donor (typically a base such as piperidine or piperazine) initiates the reaction and forms a Michael Adduct with the protecting group. Formation of the Michael Adduct leads to an intramolecular rearrangement that cleaves the protecting group from the amino acid.

The Michael addition process eliminates DBF; thus DBF never raises an issue. As a result, the amount of base can be selected as needed for cleavage rather than in order to provide a large excess for scavenging DBF. Additionally, Michael addition is more reactive than the beta elimination steps of an Fmoc deprotection. This also provides the opportunity to use a weaker base (pKa) or a lower concentration of a stronger base. In turn, in an aqueous environment, a smaller amount of base is preferred because it will tend to form less hydroxide ion (OH), minimize base catalyzed side reactions during deprotection, lower reagent costs, and reduce waste toxicity

As another advantage, the α,β group that is deblocked through a Michael addition is more base-labile than are conventional beta-elimination groups such as Fmoc. This in turn reduces or eliminates side reactions and is likewise advantageous for the aqueous solvent system because it also minimizes or eliminates the production of hydroxide ion.

In the Michael Addition mechanism the deprotection also serves as the scavenging action so that no reactive intermediate is present to react with the free amine group. The Bsmoc group is also more reactive to attack by secondary amines than the Fmoc group. These factors likewise lower the strength or concentration of the base needed in the Bsmoc deprotection reaction.

Enhanced Water Solubility

As compared to Fmoc, the structure of Bsmoc appears more soluble in water based upon its heterocyclic 5-membered ring that has an SO₂ group present. Bsmoc appears to be more soluble because it contains only one additional six-membered carbon ring. A comparison between an Fmoc and Bsmoc compound has been observed in rapid solution phase synthesis. In this type of synthesis, TAEA (tris(2-aminoethyl)amine) is used for deprotection and its adduct with Bsmoc is soluble in water, while its adduct with Fmoc is not.

The potential water soluble methods for the Bsmoc reagent can be performed with or without assistance of microwave energy.

Monitoring Capabilities of Bsmoc

The sulfone-containing protecting groups described herein (e.g., Bsmoc) present opportunities for monitoring after completion of either or both of the deprotection and coupling reactions. The single SO₂ group in these compounds is unique to most, and in many calls all, of the other reagents used during the step-wise assembly of the peptide. This SO₂ group can be monitored by infrared radiation (IR) to determine the quantitative amounts of Bsmoc (or Nsmoc, Bspoc or Mspoc) present at the end of each reaction. Evidence of the SO₂ group can be used to determine an incomplete removal of Bsmoc at the end of the deprotection. This is advantageous to the UV approach in that it does not require performing the reaction twice to make a comparison.

The coupling reaction can be monitored by IR absorption in two possible ways. The first method is to determine the IR absorption immediately after addition of the amino acid and activator reagents. This provides a baseline for total Bsmoc (Nsmoc, Bspoc, Mspoc) in the reaction vessel at the user defined excess. At the conclusion of the coupling reaction and subsequent washing the IR absorption is then again determined and compared to the initial value (addition of pure solvent in identical volume to amino acid activator solution may be necessary for comparison). A 100% complete coupling reaction should yield an IR absorption ratio that is proportional to the excess used. This approach is advantageous because it only requires the coupling reaction to be performed one time. A second approach could make a comparison of the IR absorption after two subsequent coupling reactions in a manner identical to that currently used by UV for monitoring the Fmoc deprotection step.

Side Chain Protecting Groups:

In solid phase peptide synthesis, the acids generally carry side chain protecting groups and such sidechain protected acids can be used in accordance with the present invention. Common (typical) amino acid side chain protecting groups include the following examples. Trityl- (Trt) is typically used on Cysteine (Cys), Histidine (His), Asparagine (Asn), and Glutamine (Gln). tert-Butyl (tBu) is typically used on Aspartic Acid (Asp), Glutamic Acid (Glu), Serine (Ser), Threonine (Thr), and Tyrosine (Tyr). tert-butoxycarbonyl (Boc) is typically used on Lysine (Boc), and Tryptophan (Trp). 2,2,4,6,7-Pentamethyldihydrobenzofuran-5-sulfonyl (Pbf) is typically used on Arginine (Arg). Dimethylcyclopropyl (Dmcp) is less common, but published in the literature for Fmoc derivatives.

All of these side chain protecting groups are non-polar. The Pbf group contains some polarity with the sulfonyl group, but is still largely non-polar.

The skilled person will understand that the invention includes numerous possibilities, any of which can be carried out by the skilled person and without undue experimentation. Thus, the deprotection can be carried out using amino acids protected with the Michael addition acceptor compounds, including, but not limited to Bsmoc, Nsmoc, Bspoc and Mspoc. Any one or more (or all) of the deprotection, washing, activation, coupling or cleaving steps can be carried out in water or in a water-alcohol system, with or without a detergent. Any one or more (or all) of these steps can likewise be enhanced by applying microwave irradiation.

### Example 1

In order to evaluate and demonstrate the advantages of the invention, a synthesis of the 65-74 segment of acyl carrier peptide (ACP) was carried out using Fmoc protected acids and water as the washing solvent.

Table 1 summarizes some of the results (“DMF” is dimethyl formamide, “DIC” is diisopropylcarbodiimide and HOBt is hydroxybenzotriazole).

TABLE 1

| Step | Conditions | Amount |
|---|---|---|
| Deprotection | 20% Piperidine in DMF | 7 mL |
| MW Reaction | 75° C. | 3 min |
| Wash | 7 mL H₂O | X 5 |
| Coupling | Fmoc-Amino Acid/DIC/HOBt | 4 mL |
| | (5:5:5) | |
| MW Reaction | 75° C. | 5 min |
| Wash | 7 mL H₂O | X 5 |

Using this protocol, the products clogged the synthesizer after only two cycles and further cycles could not be carried out. Because Fmoc protected acids are insoluble in water, the observed precipitation was entirely expected. It appears that automated synthesis of a peptide under this protocol would be difficult or impossible.

### Example 2

In a second experiment, the same composition as Example 1 (65-74 ACP) was synthesized using conditions otherwise identical to Example 1, but with Bsmoc protected acids instead of Fmoc protected acids. The side chain protecting groups on the amino acids were otherwise conventional, and the following amino acid derivatives were used (*DCHA refers to the dicyclohexylamine salt form of the amino acids):

Bsmoc-Gly-OH

Bsmoc-Asn(Dmcp)-OH.DCHA

Bsmoc-11e-OH.DCHA

Bsmoc-Asp(tBu)-OH.DCHA

Bsmoc-Tyr(tBu)-OH

Bsmoc-Ala-OH

Bsmoc-Gln(Dmcp)-OH.DCHA

Bsmoc-Val-OH

Table 2 summarizes these results.

TABLE 2

| Step | Conditions | Amount |
|---|---|---|
| Deprotection | 20% Piperidine in DMF | 7 mL |
| MW Reaction | 75° C. | 3 min |
| Wash | 7 mL H₂O | X 5 |
| Coupling | Bsmoc-Amino | 4 mL |
| | Acid/DIC/HOBt (5:5:5) | |
| MW Reaction | 75° C. | 5 min |
| Wash | 7 mL H₂O | X 5 |

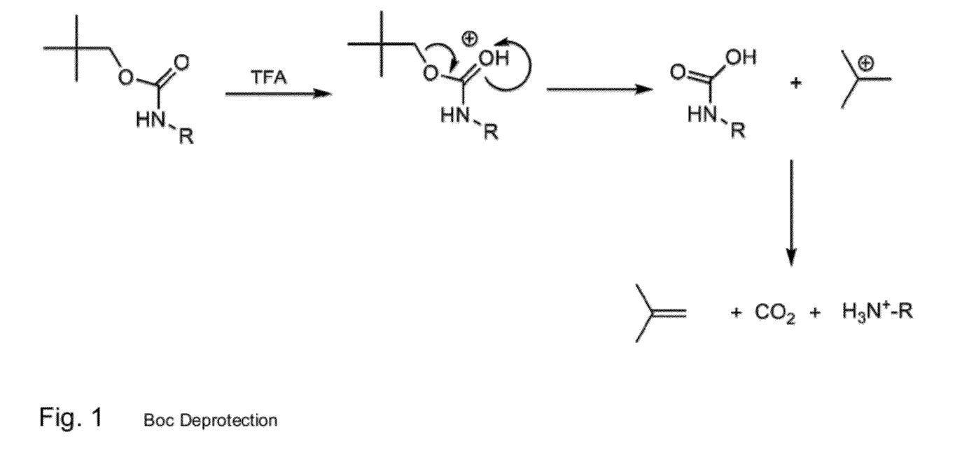

Following synthesis, the reaction product was separated by high-pressure liquid chromatography (HPLC) followed by mass spectroscopy of the relevant fraction from HPLC. FIG. 1 is a plot of the HPLC fractions and FIG. 2 is the mass spectrum of the fraction of interest.

FIG. 1 illustrates successful synthesis, but with less than desirable purity, particularly based upon the presence of significant aspartimide related side products. These side products are presumed to result from the presence of hydroxide ion (OH—) which in turn is generated by the reaction between piperidine and water.

The desired peptide appears to be the fraction just beyond 7.58 min. in FIG. 1. FIG. 2 is the mass spectrum of that fraction, and confirms the success of the synthesis, even if at lower purity.

### Example 3

In a third experiment, the 65-74 ACP synthesis was carried out with water washing and Bsmoc protected acids, but using a five percent (5%) concentration of piperazine as the base rather than the 20% concentration of piperidine used in Example 2. Relevant data is summarized in Table 3.

TABLE 3

| Step | Conditions | Amount |
|---|---|---|
| Deprotection | 5% Piperazine w/0.1M HOBt | 7 mL |
| | in DMF | |
| MW Reaction | 75° C. | 3 min |
| Wash | 7 mL H₂O | X 5 |
| Coupling | Bsmoc-Amino | 4 mL |
| | Acid/DIC/HOBt (5:5:5) | |
| MW Reaction | 75° C. | 5 min |
| Wash | 7 mL DMF | X 1 |
| Wash | 7 mL H₂O | X 4 |

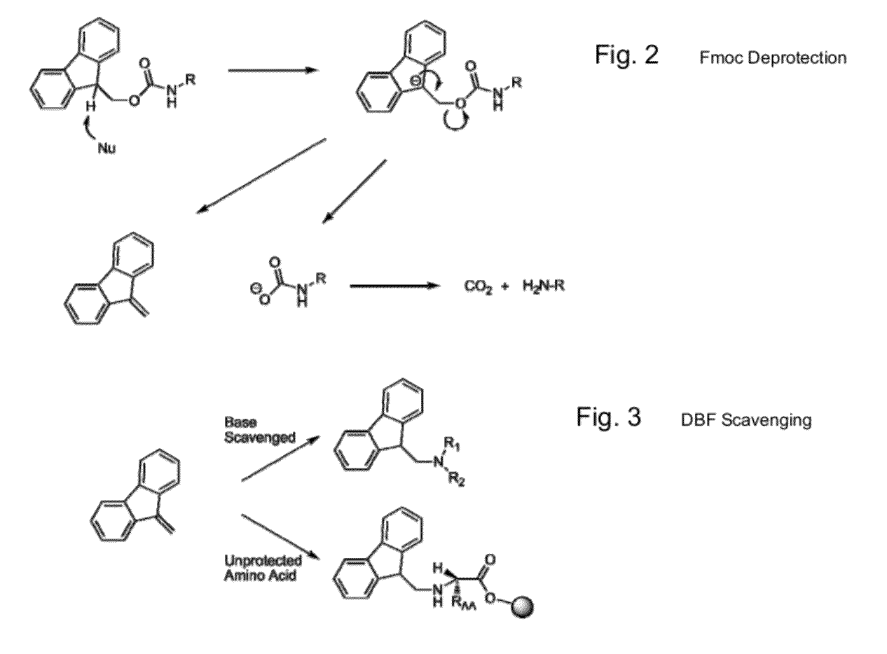

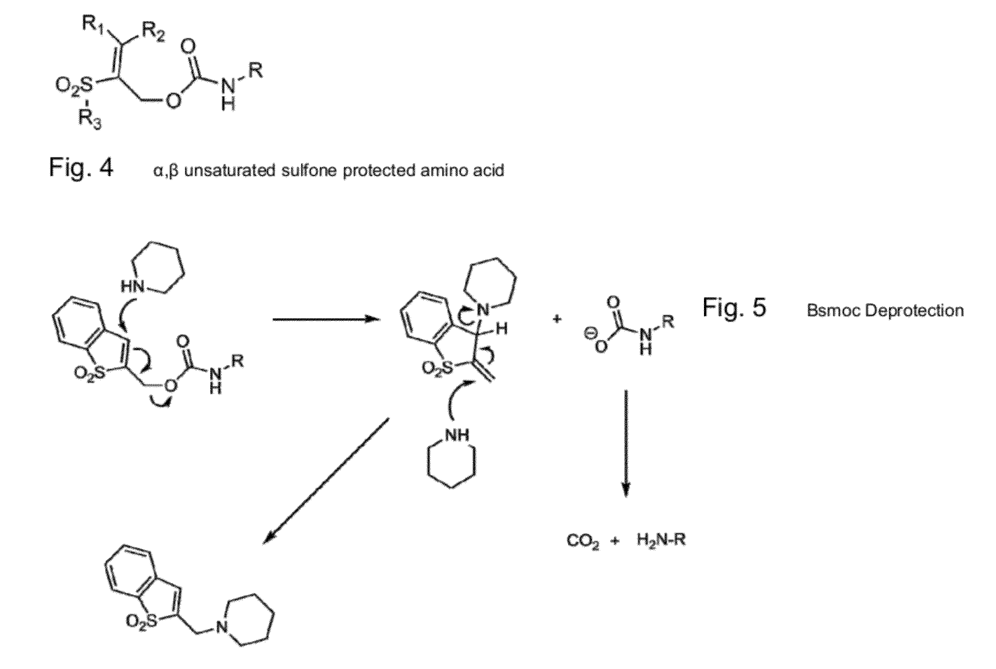

FIGS. 3 and 4 further demonstrate some of the results. FIG. 3 is another HPLC plot and FIG. 4 is the mass spectrum of the relevant fraction taken from the HPLC. The results show that the purity of the product was higher than Example 2, presumably because the weaker base piperazine reduces (as compared to piperidine) the formation of the hydroxide ion.

### Example 4

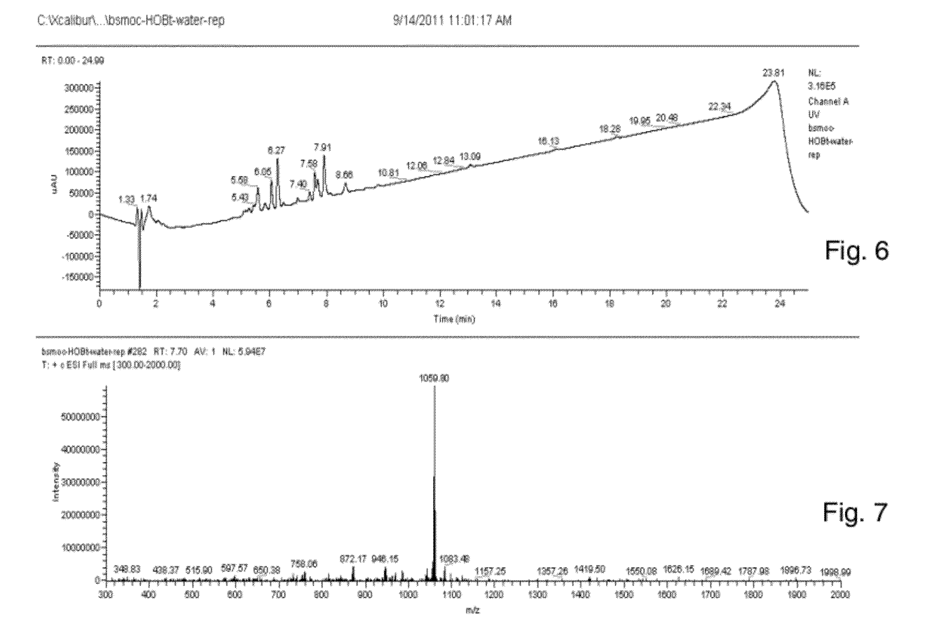

As a fourth experiment, the 65-74 ACP synthesis was carried out in the same manner as Example 3, but with the addition of an alcohol wash (isopropyl alcohol) immediately before the coupling step. The results are summarized in Table 4 and the HPLC and mass spectrum are shown in FIGS. 5 and 6.

TABLE 4

| Step | Conditions | Amount |
|---|---|---|
| Wash | 7 mL IPA | X 1 |
| Deprotection | 3% Piperazine w/0.1M HOBt | 7 mL |
| | in DMF | |
| MW Reaction | 75° C. | 3 min |
| Wash | 7 mL IPA | X 1 |
| Wash | 7 mL H₂O | X 3 |
| Wash | 7 mL IPA | X 1 |
| Coupling | Bsmoc-Amino | 4 mL |
| | Acid/DIC/HOBt (5:5:5) | |
| MW Reaction | 75° C. | 5 min |
| Wash | 7 mL IPA | X 1 |
| Wash | 7 mL H₂O | X 3 |

Table 4 and FIGS. 5 and 6 illustrate that the peptide was successfully synthesized at high purity using this approach. In fact, the purity was as high as any observed with conventional organic solvents (e.g., DMF, N-methylpyrrolidone) for the washing steps. The isopropyl alcohol wash prior to the coupling reaction appears to be helpful in reducing hydrolysis of the activated ester thus leading to higher coupling efficiency.

### Example 5

As a fifth experiment, the 65-74 ACP synthesis was carried out using Bsmoc protected amino acids, piperazine as the base, and water as the washing solvent, but under conventional (i.e., other than microwave-assisted) conditions.

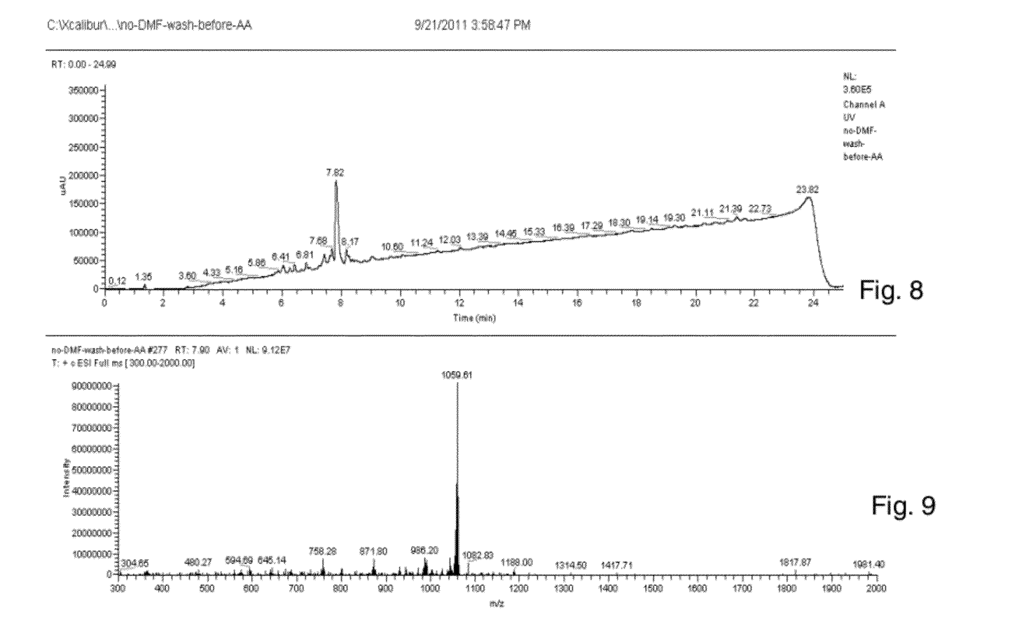

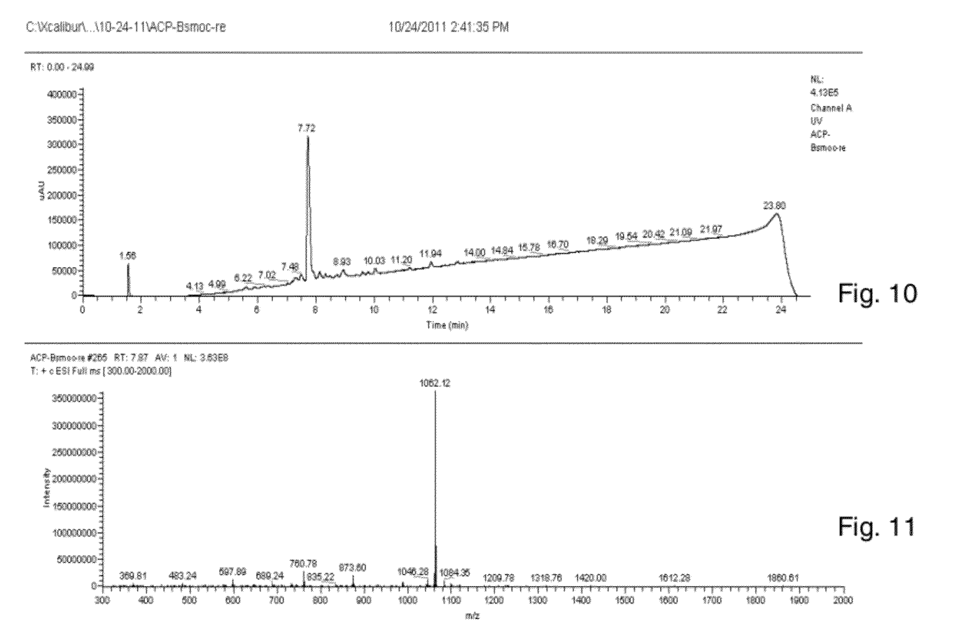

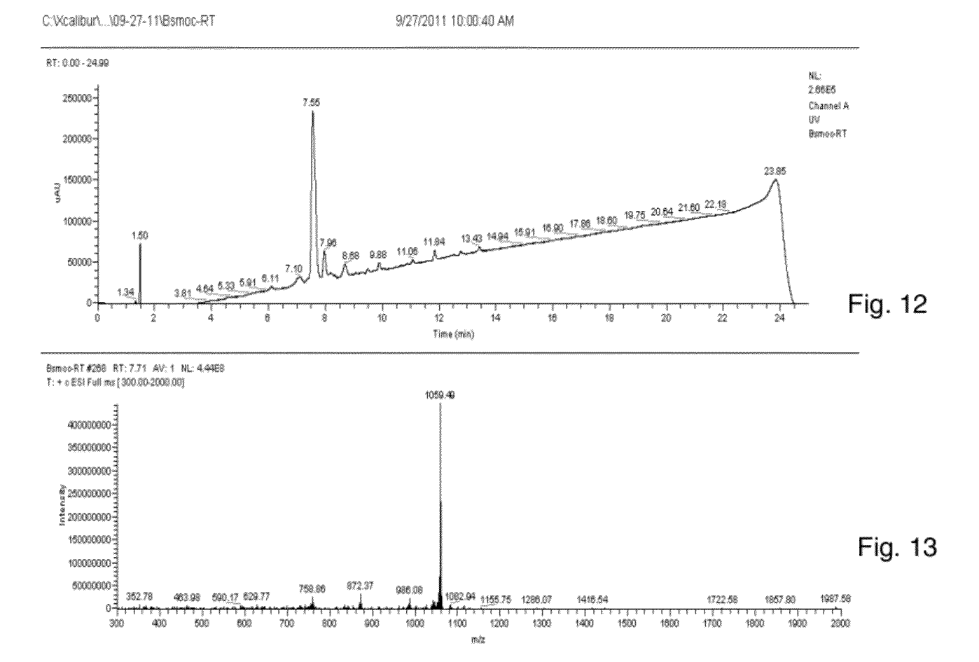

Table 5 and FIGS. 7 and 8 illustrate the results. As with the previous experiments, FIG. 7 is a plot of the HPLC fractions and FIG. 8 is the mass spectrum of the relevant fraction.

As an explanatory note, because of a slightly improper calibration of the mass spectrometer, the peaks labeled at 1059 in FIGS. 7, 9 and 13 actually represented the 1062 fragment (e.g., FIG. 11).

TABLE 5

| Step | Conditions | Amount |
|---|---|---|
| Deprotection | 5% Piperazine w/0.1M HOBt | 7 mL |
| | in DMF | |
| Reaction | Room Temperature | 10 min |
| Wash | 7 mL H₂O | X 4 |
| Wash | 7 mL DMF | X 1 |
| Coupling | Bsmoc-Amino | 4 mL |
| | Acid/DIC/HOBt (5:5:5) | |
| Reaction | Room Temperature | 30 min |
| Wash | 7 mL DMF | X 1 |
| Wash | 7 mL H₂O | X 4 |

*A double coupling was used for Asn.

The information in Table 5 and FIGS. 7 and 8 demonstrate that the peptide was successfully synthesized in high purity using this protocol. A minor deletion fraction did appear at 7.96 min. These results show that the advantages of the water or water-alcohol washing using Bsmoc protected amino acids can be performed successfully using either microwave assisted SPPS or conventional SPPS. Indeed, these conditions were not optimized because two DMF washes were still included, but the results illustrate the feasibility of the method.

Such feasibility under conventional conditions is expected to be quite valuable for scale up techniques.

In the specification there have been set forth preferred embodiments of the invention, and although specific terms have been employed, they are used in a generic and descriptive sense only and not for purposes of limitation, the scope of the invention being defined in the claims.

1. In a solid phase peptide synthesis method, the improvement comprising
deprotecting an amino group in its protected form that is protected with a protecting group containing an α,β-unsaturated sulfone; and
washing the deprotected acid in a solvent selected from the group consisting of water, alcohol, and mixtures of water and alcohol.

2. A method according to claim 1 wherein the α,β-unsaturated sulfone protecting group acts as a Michael acceptor site.

3. A method according to claim 1 comprising washing the deprotected acid in a solvent that also includes a detergent.

4. A method according to claim 1 comprising deprotecting an amino acid that is soluble in water in its protected form and that is protected with a protecting group containing an α,β-unsaturated sulfone.

5. A method according to claim 1 wherein the protecting group is selected from the group consisting of Bsmoc, Nsmoc, Bspoc and Mspoc.

6. A method according to claim 1 in which the protecting group is Bsmoc and the washing solvent is water.

7. A method according to claim 1 further comprising irradiating the deprotected acid and the solvent with microwave irradiation during the washing step.

8. A method according to claim 1 wherein the washing step is carried out in a mixture of water and alcohol and wherein the alcohol is selected from the group consisting of methanol, ethanol, 1-propanol, 2-propanol, n-butanol, isobutanol, sec-butanol, and tert-butanol.

9. A method according to claim 1 further comprising coupling the washed deprotected acid to a second acid that is also protected with a protecting group containing an α,β-unsaturated sulfone.

10. A method according to claim 9 comprising adding the second amino acid with an activating solution.

11. A method according to claim 9 further comprising washing the coupled acids in a solvent selected from the group consisting of water, alcohol, and mixtures of water and alcohol.

12. A method according to claim 11 further comprising
deprotecting the second protected amino acid;
washing the deprotected second acid and other compositions in a solvent selected from the group consisting of water, alcohol, and mixtures of water and alcohol;
coupling the deprotected second amino acid to a third protected acid in which the third acid is also protected with (describe); and
washing the coupled acids and solid phase resins with a solvent selected from the group consisting of water, alcohol, and mixtures of water and alcohol.

13. A method according to claim 12 comprising repeating the steps of deprotecting, washing, coupling, and washing for fourth and successive acids to produce a desired peptide chain.

14. In a solid phase peptide synthesis method, the improvement comprising
coupling a deprotected amino acid that in its protected form is protected with a protecting group containing an α,β-unsaturated sulfone; and
washing the coupled acid in a solvent selected from the group consisting of water, alcohol, and mixtures of water and alcohol.

15. A method according to claim 14 wherein the α,β-unsaturated sulfone protecting group acts as a Michael acceptor site.

16. A method according to claim 14 comprising washing the deprotected acid in a solvent that also includes a detergent.

17. A method according to claim 14 comprising coupling an amino acid that is soluble in water in its protected form.

18. A method according to claim 14 wherein the protecting group is selected from the group consisting of Bsmoc, Nsmoc, Bspoc and Mspoc.

19. A method according to claim 14 in which the protecting group is Bsmoc and the washing solvent is water.

20. A method according to claim 14 further comprising irradiating the deprotected acid and the solvent with microwave irradiation during the washing step.

21. A method according to claim 14 wherein the washing step is carried out in a mixture of water and alcohol and wherein the alcohol is selected from the group consisting of methanol, ethanol, 1-propanol, 2-propanol, n-butanol, isobutanol, sec-butanol, and tert-butanol.

22. A method according to claim 14 comprising coupling the amino acid in the presence of an activating solution.

23. A composition comprising:
a solid phase resin;
an amino acid protected with a protecting group containing an α,β-unsaturated sulfone;
a solvent selected from the group consisting of water, alcohol, and mixtures of water and alcohol;
a base for deprotecting said protected amino acid; and
the adduct formed by the reaction between said deprotecting base and said α,β-unsaturated sulfone protecting group.

24. A composition according to claim 23 wherein said protecting group includes a Michael acceptor site.

25. A composition according to claim 23 wherein said protecting group is selected from the group consisting of Bsmoc, Nsmoc, Bspoc, and Mspoc.

26. A composition according to claim 23 wherein said deprotecting base is water soluble.

27. A composition according to claim 23 wherein said base is selected from the group consisting of piperazine and morpholine.

28. A composition according to claim 23 wherein said solvent is a mixture of alcohol and water and said alcohol is selected from the group consisting of methanol, ethanol, 1-propanol, 2-propanol, n-butanol, isobutanol, sec-butanol, and tert-butanol.

29. A composition according to claim 23 wherein said solid phase resin is selected from the group consisting of polyethylene glycol resins and polyethylene glycol resins spacer resins.

30. A composition comprising
a solid phase resin;
a solvent selected from the group consisting of water, alcohol, and mixtures of water and alcohol;
an unactivated α,β-unsaturated sulfone based protected amino acid; and
activated portions of said α,β-unsaturated sulfone based protected amino acid.

31. A composition according to claim 30 wherein said protecting group includes a Michael acceptor site.

32. A composition according to claim 30 wherein said protecting group is selected from the group consisting of Bsmoc, Nsmoc, Bspoc, and Mspoc.

33. A composition according to claim 30 wherein said solvent is a mixture of alcohol and water and said alcohol is selected from the group consisting of methanol, ethanol, 1-propanol, 2-propanol, n-butanol, isobutanol, sec-butanol, and tert-butanol.

34. A composition according to claim 30 wherein said solid phase resin is selected from the group consisting of polyethylene glycol resins and polyethylene glycol resins spacer resins.

35. A solid phase peptide synthesis method that includes the steps of:
deprotecting an amino group in its protected form that is protected with a protecting group containing an alpha, beta unsaturated sulfone; and
washing the deprotected acid in a solvent selected from the group consisting of water, alcohol, and mixtures of water and alcohol.

36. A solid phase peptide synthesis method according to claim 35 and further comprising:
coupling the deprotected acid to a resin-based peptide or a resin-based amino acid; and
washing the coupled composition in a solvent selected from the group consisting of water, alcohol, and mixtures of water and alcohol.

37. A solid phase peptide synthesis method according to claim 35 comprising deprotecting an amino group in its protected form that is protected with a protecting group that includes a Michael acceptor site.

38. A solid phase peptide synthesis method according to claim 36 comprising washing the compositions with alcohol prior to the coupling step.

39. A solid phase peptide synthesis method according to claim 38 comprising washing the compositions with an alcohol selected from the group consisting of methanol, ethanol and propanol.

40. A solid phase peptide synthesis method according to claim 35 comprising deprotecting the protected acid in the presence of a weak base.

41. A solid phase peptide synthesis method according to claim 40 comprising deprotecting the protected acid in the presence of a base selected from the group consisting of piperidine and piperazine.

42. A solid phase peptide synthesis method that includes the steps of:
deprotecting an amino group in its protected form that is protected with a protecting group containing an alpha, beta unsaturated sulfone;
washing the deprotected acid in a solvent selected from the group consisting of water, alcohol, and mixtures of water and alcohol.
coupling the deprotected acid to a resin-based peptide or a resin-based amino acid; and
washing the coupled composition in a solvent selected from the group consisting of water, alcohol, and mixtures of water and alcohol.

43. A method according to claim 42 comprising irradiating the compositions with microwaves during at least the deprotecting and coupling steps.

44. A method according to claim 42 comprising conductively heating the compositions during at least the deprotecting and coupling steps.

45. A method according to claim 42 comprising washing the protected acid and the solid phase resin with alcohol prior to the deprotection step.

46. A method according to claim 42 wherein the step of washing the deprotected acid comprises washing with propanol.

47. A method according to claim 42 wherein the step of washing the deprotected acid comprises the steps of washing with alcohol, then washing with water, and then washing with alcohol a second time.

48. A method according to claim 42 wherein the step of washing the coupled composition comprises washing with alcohol.

49. A method according to claim 42 wherein the step of washing the coupled composition comprises washing with propanol and then washing with water.

50. In a solid phase peptide synthesis method, the improvement comprising:
monitoring the SO₂ group of an α,β-unsaturated sulfone that protects an amino acid in the synthesis by infrared radiation to determine the quantitative amounts of the α,β-unsaturated protecting group present at the end of a step selected from the group consisting of the deprotecting reaction and the coupling reaction.

51. A method according to claim 50 in which the α,β-unsaturated sulfone is selected from the group consisting of Bsmoc, Nsmoc, Bspoc and Mspoc.

52. A method according to claim 50 comprising monitoring the coupling reaction by:
determining the infrared absorption prior to addition of the protected amino acid and activator reagents;
thereafter determining the infrared absorption at the conclusion of the coupling reaction and any subsequent washing; and
comparing the two absorptions.

53. A method according to claim 50 comprising monitoring the coupling reaction by:
measuring infrared absorption after each of two consecutive coupling reactions; and
comparing the measured infrared absorptions.

54. A method according to claim 50 comprising monitoring the deprotection reaction by:
measuring infrared absorption after each of two deprotection reactions; and
comparing the measured infrared absorptions.
